(19) United States
(12) Patent Application Publication
(10) Pub. No.: US 2020/0254187 A1
(43) Pub. Date: Aug. 13, 2020

(54) CAP FOR DISINFECTION OF A MEDICAL DEVICE

(71) Applicant: Becton, Dickinson and Company, Franklin Lakes, NJ (US)

(72) Inventors: Maryann Fam, Secaucus, NJ (US); Peter Girgis, East Brunswick, NJ (US); Jeremy Gartner, Wyckoff, NJ (US)

(73) Assignee: Becton, Dickinson and Company, Franklin Lakes, NJ (US)

(21) Appl. No.: 16/777,553

(22) Filed: Jan. 30, 2020

Related U.S. Application Data
(60) Provisional application No. 62/804,415, filed on Feb. 12, 2019.

(51) Int. Cl. A61M 5/32

(52) U.S. Cl. CPC A61M 5/3202; A61M 2005/3254; A61M 2205/368; A61M 2205/3306; A61M 2205/8206; A61M 2205/273; A61M 2205/50; A61M 2205/051

(57) ABSTRACT

A cap configured to disinfect a medical device or portion thereof, the cap comprising a power source that provides electric power, an electromagnetic radiation source that uses the electric power received from the power source to emit photons for disinfection and a switch that is configured to be operated by user action, wherein upon activation of the switch, the electric power from the power source is applied to the electromagnetic radiation source to radiate photons onto the medical device or portion thereof.

### RELATED APPLICATIONS

This application claims the benefit of U.S. Provisional Patent Application Ser. No. 62/804,415 filed on Feb. 12, 2019, which is hereby incorporated by reference.

### FIELD OF THE INVENTION

The present invention relates to a cap that disinfects a medical device or a portion thereof, such as a septum of a medication delivery pen.

### BACKGROUND OF THE INVENTION

Insulin and other injectable medications are commonly given with a medical device, such as a drug delivery device or a medication delivery pen, whereby a disposable pen needle is attached to facilitate drug container access and allow fluid egress from the container, through the needle and into the patient.

As technology and competition advance, driving the desire for shorter, thinner, less painful, and more efficacious injections, the design of the pen needle and parts thereof becomes more and more important. Designs need to proactively address ergonomically improving injection technique, injection depth control and accuracy, the ability to be safely used and transported for disposal, sterilization, disinfection, and protection against misuse while maintaining the ability to be economically manufactured on a mass production scale.

Drug delivery devices, such as the exemplary medication delivery pen 10 shown in FIGS. 1 and 2, can be designed for subcutaneous as well as intradermal injections, and typically comprise a dose knob/button 22, an outer sleeve or housing 11, and a cap 50. The dose knob/button 22 allows a clinician or patient to set the dosage of medication to be injected. The housing 11 is gripped by the user when injecting medication. The cap 50 can be used by the user to securely hold the medication delivery pen 10 in a shirt pocket, purse or other suitable location and provide cover/protection from accidental needle injury. The cap 50 is also used to cover a septum 18 of the medicament cartridge 16 in the medication delivery pen 10 before and after use. Otherwise, the septum 18 would be exposed.

FIG. 2 is an exploded view of the medication delivery pen 10 of FIG. 1. The dose knob/button 22 has a dual purpose and is used both to set the dosage of the medication to be injected and to inject the dosed medicament via the leadscrew 12 and plunger/stopper 14 through the medicament cartridge 16, which is attached to the medication delivery pen 10 through a body 20. In standard medication delivery pens, the dosing and delivery mechanisms are all found within the housing 11 and are not described in greater detail here as they are understood by those knowledgeable of the prior art.

For operation, the medication delivery pen 10 is attached to a pen needle comprising a needle/cannula 30, a septum penetrating cannula 32 and a hub 34. Specifically, a distal movement of the plunger or stopper 14 within the medicament cartridge 16 causes medication to be forced into the needle 30 of the hub 34. The medicament cartridge 16 is sealed by the septum 18, which is punctured by the septum penetrating needle cannula 32 located within the hub 34. The hub 34 is preferably screwed onto the body 20, although other attachment means can be used.

To protect a user from accidental needle sticks, or anyone who handles the pen needle, an outer cover 38, which attaches to the hub 34, covers the hub 34. An inner shield 36 covers the patient needle 30 within the outer cover 38. The inner shield 36 can be secured to the hub 34 to cover the patient needle 30 by any suitable means, such as an interference fit or a snap fit. The outer cover 38 and the inner shield 36 are removed prior to use.

The medicament cartridge 16 is typically a glass tube or vial sealed at one end with the septum 18 and sealed at the other end with the stopper 14. The septum 18 is pierceable by a septum penetrating cannula 32 in the hub 34, but does not move with respect to the medicament cartridge 16. The stopper 14 is axially displaceable within the medicament cartridge 16 while maintaining a fluid tight seal.

Existing medication delivery pens are disclosed in U.S. Patent Application Publication Nos. 2006/0229562 to Marsh et al., which was published on Oct. 12, 2006, and 2007/0149924 to R. Marsh, which was published on Jun. 28, 2007, the entire contents of both of which are hereby incorporated by reference for this purpose.

Medical devices such as medication delivery pens 10 are typically prepared for use by disinfecting the septum 18 with an alcohol swab prior to attaching the pen needle for medication delivery. However, challenges arise when using medication delivery pens 10 for patient care. Carrying alcohol swabs with the medication delivery pens 10 can be burdensome for a user. In certain circumstances, the septum 18 may not be properly disinfected prior to use. Thus, an improved disinfecting device and process for use with medication delivery pens 10 is desired.

### SUMMARY OF THE INVENTION

It is an aspect of the present invention to provide a cap, that disinfects a medical device or a portion thereof, such as a septum surface. Such a configuration improves workflow and convenience of users using various medical devices such as pen injectors. Poor injection practice is minimized since the user is no longer relied upon to disinfect the septum or other exposed surface or portion with an alcohol swab. In fact, the cap can be configured to automatically disinfect the septum or other exposed surface or portion, thus saving time. Disinfecting the medical device with the cap is also more controlled or automated to satisfy high accuracy and performance requirements. Finally, the user no longer needs to carry alcohol swabs for the medical device.

The foregoing and/or other aspects of the present invention can be achieved by providing a cap configured to disinfect a medical device or portion thereof, the cap comprising a power source that provides electric power, an electromagnetic radiation source that uses the electric power received from the power source to emit photons for disinfection and a switch that is configured to be operated by user action, wherein upon activation of the switch, the electric power from the power source is applied to the electromagnetic radiation source to radiate photons onto the medical device.

The foregoing and/or other aspects of the present invention can further be achieved by providing a cap configured to disinfect a medical device or portion thereof, the cap comprising a power source that provides power to a microcontroller, the microcontroller sensing and controlling the operation of the cap, an electromagnetic radiation source that radiates photons on the medical device for disinfection under control of the microcontroller, and a switch that causes the microcontroller to activate and deactivate the electromagnetic radiation source.

The foregoing and/or other aspects of the present invention can also be achieved by providing a method for disinfecting a medical device or portion thereof with a cap, the method comprising disposing an electromagnetic radiation source on an inner surface of the cap, securing the cap to the medical device, activating the electromagnetic radiation source to emit photons to disinfect the medical device, and exposing the medical device to photons from the electromagnetic radiation source.

Additional and/or other aspects and advantages of the present invention will be set forth in the description that follows, or will be apparent from the description, or may be learned by practice of the invention.

### BRIEF DESCRIPTION OF THE DRAWINGS

The above aspects and features of the present invention will be more apparent from the description for the exemplary embodiments of the present invention taken with reference to the accompanying drawings, in which:

FIG. 1 is a perspective view of an assembled medication delivery pen of the prior art;

FIG. 2 is an exploded perspective view of the components of the medication delivery pen of FIG. 1 and of a pen needle;

FIG. 3 is an exemplary embodiment of a cross-sectional view of a cap of the medication delivery pen;

FIG. 4 is a schematic drawing of the electrical components within the cap of FIG. 3 without user input;

FIG. 5 is a schematic drawing of the electrical components within the cap of FIG. 3 with user input; and

FIG. 6 is a schematic diagram of an electrical circuit of another exemplary embodiment of the cap.

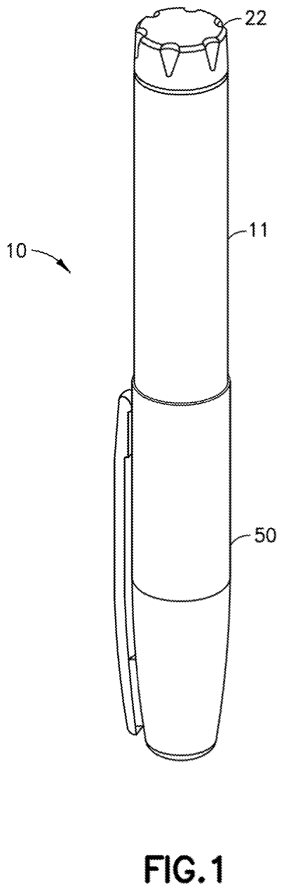

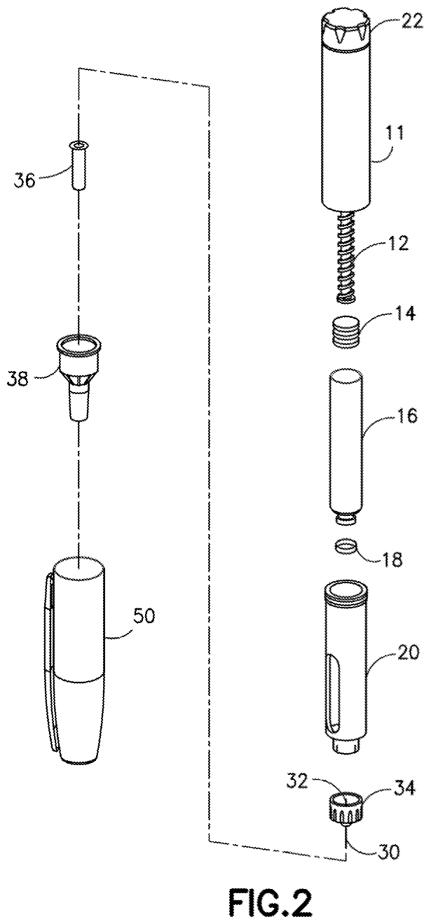

### DETAILED DESCRIPTION OF EXEMPLARY EMBODIMENTS

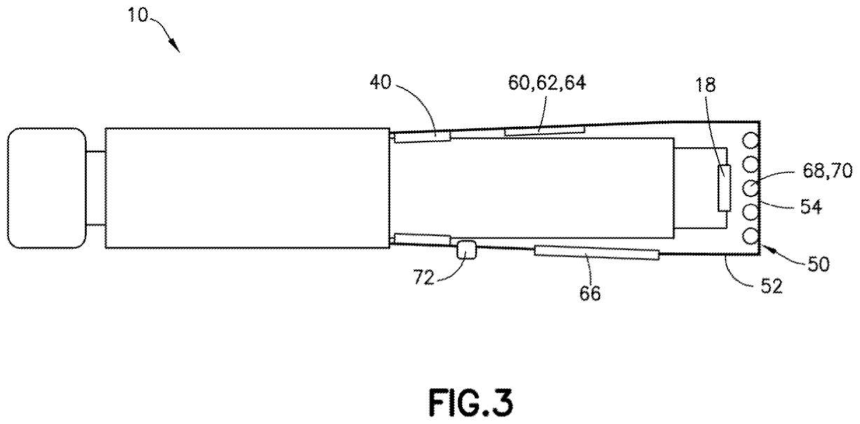

FIG. 3 illustrates a cap 50 for a medical device such as a pen injector in accordance with an embodiment of the present invention. The cap 50 includes a sidewall 52 and a top wall 54. The cap 50 is configured to enclose a distal portion of the medication delivery pen 10. Specifically, when the cap 50 is mounted onto the medication delivery pen 10, the top wall 54 is positioned opposing the septum 18 of the medication delivery pen 10. The sidewall 52 is connected to the top wall 54 and encircle the body 20. In this configuration, a distal end of the cap 50 is disposed substantially central in a longitudinal axis of the medication delivery pen 10.

The embodiments of the cap 50 disclosed herein are most commonly configured to mount on to the medication delivery pen 10 without the pen needle present. However, with appropriate modifications, other types of medical devices such as needleless IV connectors, extension sets, IV sets, catheters, syringes (such as pre-fillable syringes), medication (e.g. insulin) vials, and other devices with externally accessible surfaces that require disinfection can incorporate the cap 50 for disinfection purposes. Any surface or portion of the medical device that is contained within the cap 50 and exposed to the electromagnetic radiation source 68 can be disinfected.

With respect to the medication delivery pen 10, the operation of the cap 50 can still take place even if the pen needle is attached to the medication delivery pen 10 and covered by the cap 50. In this scenario, the pen needle can be disinfected rather than the septum 18. However, this condition is typically not preferred because it is not advisable to reuse pen needles.

The cap 50 is configured to be connectable to the pen injector 10 either indirectly via a universal fitting 40 (illustrated in FIGS. 3-5) or directly without the universal fitting 40 (not shown). Exemplary embodiments of the universal fitting 40 include a ring that tightens the fit between a distal end of the cap 50 and the medicament cartridge 16 of the medication delivery pen 10. A rotating sleeve that reduces the inner diameter when rotated and acts similarly to a telescoping pole is another universal fitting 40 that tightens the fit between the cap 50 and the medication delivery pen 10. Further, using ribs, pleats, or scales as the universal fitting 40 provides an expandable, contractible and/or friction surface at the interface between the distal end of the cap 50 and the body 20. The universal fitting 40 can have prongs to provide a mechanical engagement between the cap 50 and the body 20. Finally, another embodiment of the universal fitting 40 is a spring-loaded member that provides an applied force between a distal end of the cap 50 and the medication delivery pen 10.

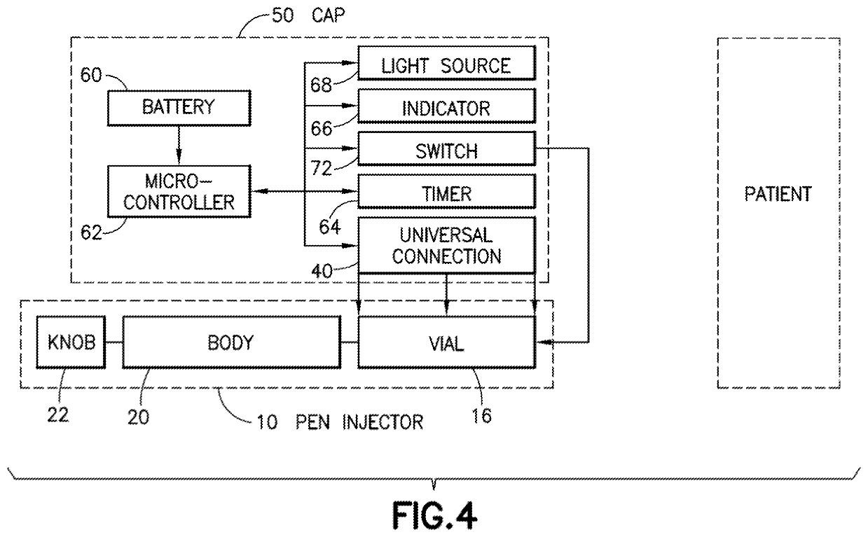

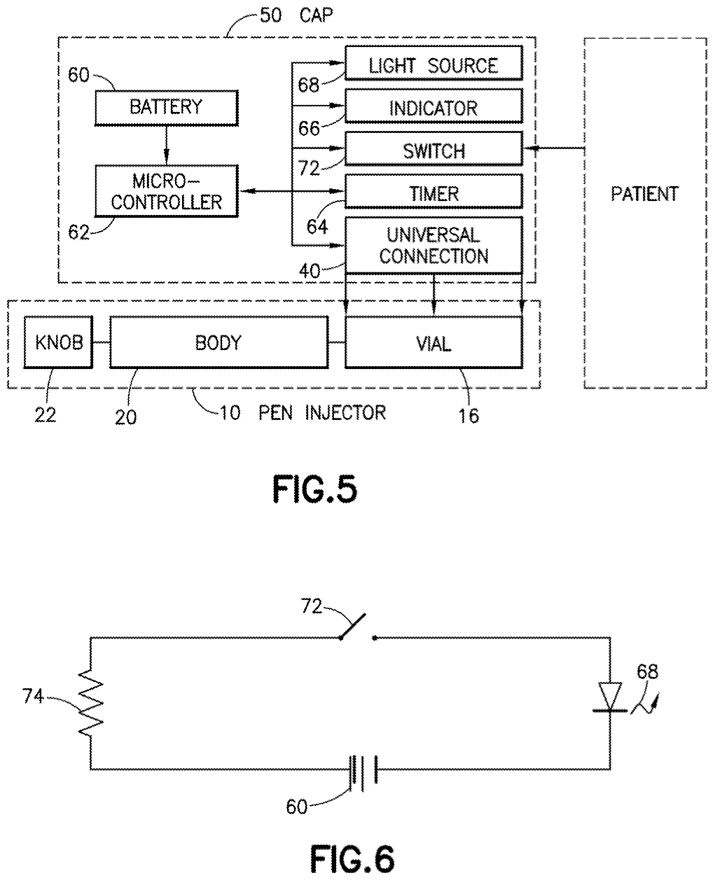

Status of the use of the universal fitting 40 is provided as feedback to a microcontroller 62, as described further below and as illustrated in FIGS. 4 and 5. Status of the use of the universal fitting 40 includes, for example, a capped position when an exterior surface of the universal fitting 40 is engaged to an interior surface of the cap 50 and when an interior surface of the universal fitting 40 is engaged to an exterior surface of the medicament cartridge 16 of the medication delivery pen 10. Status of the use of the universal fitting 40 also includes, for example, an uncapped position when one or both of these connections are disengaged. Alternately, the universal fitting 40 can be used without the cooperation of the microcontroller 62 as described further in FIG. 6.

The universal fitting 40 can also cooperate with the microcontroller 62 to vary commands for emitting photons 70 based on the status. For example, when the universal fitting 40 and the cap 50 are engaged, the microcontroller 62 issues a command for emission of photons 70. On the other hand, if one or both of the connections are disengaged, the microcontroller 62 does not issue a command for emission of photons 70.

The cap 50 includes a power source 60 that provides power to the cap 50. The power source 60 is preferably a flexible battery that wraps along an inner surface of the sidewall 52. The power source 60 can also be a lithium battery. Finally, the power source 60 can be wired circuitry that provides power (AC/DC current) to the cap 50.

If the power source 60 is a battery, the battery 60 can be rechargeable via solar energy, motion or electricity (wired or wireless). Alternatively, or in addition, the battery 60 can be discarded and replaced. Further, the cap 50 can be replaced when the battery 60 is depleted. The power source 60 can be disposed on the inner or outer surface of the sidewall 52 or top wall 54.

As illustrated in FIGS. 3-5, the power source 60 is configured to specifically provide power to the microcontroller 62 of the cap 50 or directly to an electromagnetic radiation source 68 (see FIG. 6). The electromagnetic radiation source 68 can emit electromagnetic radiation such as photons in a selected wavelength range, including ultraviolet (UV) light 70. The microcontroller 62, as commonly understood by one skilled in the art, is programmed to sense and control the operation of the cap 50. Specifically, the microcontroller 62 receives feedback and issues commands to various components of the cap 50 including, for example, the universal fitting 40 (as described above), a timer 64, an indicator 66, an electromagnetic radiation source 68 and a switch 72.

The electromagnetic radiation source 68 advantageously emits photons 70 for disinfection of the septum 18 of the medication delivery pen 10. Photons 70 are also emitted onto other surface or portion of the medication delivery pen 10 that are enclosed by the cap 50. The electromagnetic radiation source 68 is disposed on an inner surface of the top wall 54 of the cap 50.

In another embodiment, the power source 60 and the electromagnetic radiation source 68 are both stacked on the inner surface of the top wall 54 of the cap 50. Accordingly, the electromagnetic radiation source 68 is disposed distal to the power source 60 so that the photons 70 can be directly emitted on the septum 18 of the medication delivery pen 10, as well as onto other surface or portion of the medication delivery pen 10.

In a further embodiment, the electromagnetic radiation source 68 is positioned so that the photons 70 are not directly emitted on the septum 18. Although radiating photons 70 directly on the septum 18 is more efficient, such a configuration is not critical for effective operation and disinfecting.

The command that controls the operation of the electromagnetic radiation source 68 is received from the microcontroller 62 or directly from the switch 72 (see FIG. 6). The electromagnetic radiation source 68 is preferably a commercially known and available plurality of light emitting diodes (LEDs). LEDs provide advantages in emitting light in optimal wavelength(s) for improved disinfection, have a small footprint, and consume far less energy due to their instant on/off capability. Nevertheless, any energy source that disinfects can be used.

A variety of wavelength ranges from the electromagnetic spectrum can be used for disinfection. As an example, the relative effectiveness of UV light wavelengths for this process is known as the germicidal action spectrum, which peaks at a maximum wavelength 265 nm (UV-C). Thus, a preferred wavelength range of UV light 70 is between 250 nm and 280 nm. The necessary exposure for many applications ranges between 10 mJ/cm² and 100 mJ/cm².

In view of the above, alternative wavelengths may be used. All UV light wavelengths shorter than 300 nm are effective to disinfect and kill microorganisms. However, given enough energy, longer wavelengths can also be equally as effective.

The destruction of microorganisms by UV light 70 is an exponential process. The higher the given exposure, the higher the proportion of microorganisms destroyed. Consequently, the exposure necessary to destroy 99% is double the value to destroy 90%. It follows therefore that the exposure required to kill 99.9% is three times the value to destroy 90% and the exposure required to kill 99.99% is four times the value to destroy 90%.

Although a preferred wavelength range of UV light 70 is desired, the duration of emission of UV light 70 required for disinfection is a function of distance, power, time and wavelength. The required exposure (i.e. UV Dose or Energy) can be calculated using the following equation:

UV light dose (J/m2)=Irradiance (W/m2)×Exposure Time (seconds)

The necessary wavelength and exposure time can be calculated based on a required dose of UV light 70 as set forth in the following table:

### Bacteria-UV Light Dose Correlation Table:

| | UV light exposure (dose) in J/m2 | | | |
|---|---|---|---|---|
| | required to achieve 90%-99.99% | | | |
| | reduction of the specified | | | |
| | microorganism types | | | |
| | 90% | 99% | 99.9% | 99.99% |
| Microorganism (microbe) | (1 log) | (2 log) | (3 log) | (4 log) |
| Bacillus anthracis - | 45.2 | 90.40 | 135.60 | 180.80 |
| Anthrax | | | | |
| Bacillus anthracis | 243.2 | 486.40 | 729.60 | 972.80 |
| spores - Anthrax spores | | | | |
| Bacillus magaterium sp. | 27.3 | 54.60 | 81.90 | 109.20 |
| (spores) | | | | |
| Bacillus magaterium sp. | 13.0 | 26.0 | 39.0 | 52.0 |
| (veg.) | | | | |
| Bacillus paratyphusus | 32.0 | 64.0 | 96.0 | 128.0 |
| Bacillus subtilis spores | 116.0 | 232.0 | 348.0 | 464.0 |
| Bacillus subtilis | 58.0 | 116.0 | 174.0 | 232.0 |
| Clostridium difficile | 60.0 | 120.0 | 180.0 | 240.0 |
| (C. difficile or C. diff) | | | | |
| Clostridium tetani | 130.0 | 260.0 | 390.0 | 520.0 |
| Corynebacterium | 33.7 | 67.4 | 101.1 | 134.80 |
| diphtheria | | | | |
| Ebertelia typhosa | 21.4 | 42.80 | 64.2 | 85.60 |
| Escherichia coli | 30.0 | 60.0 | 90.0 | 120.0 |
| Leptospiracanicola - | 31.5 | 63.0 | 94.5 | 126.0 |
| infectious Jaundice | | | | |
| Microccocus candidus | 60.5 | 121.0 | 181.5 | 242.0 |
| Microccocus sphaeroides | 10.0 | 20.0 | 30.0 | 40.0 |
| MRSA | 32.0 | 64.0 | 96.0 | 128.0 |
| Mycobacterium | 62.0 | 124.0 | 186.0 | 248.0 |
| tuberculosis | | | | |
| Neisseria catarrhalis | 44.0 | 88.0 | 132.0 | 176.0 |
| Phytomonas tumefaciens | 44.0 | 88.0 | 132.0 | 176.0 |
| Proteus vulgaris | 30.0 | 60.0 | 90.0 | 120.0 |
| Pseudomonas aeruginosa | 55.0 | 110.0 | 165.0 | 220.0 |
| Pseudomonas fluorescens | 35.0 | 70.0 | 105.0 | 140.0 |
| Salmonella enteritidis | 40.0 | 80.0 | 120.0 | 160.0 |
| Salmonela paratyphi - | 32.0 | 64.0 | 96.0 | 128.0 |
| Enteric fever | | | | |
| Salmonella typhosa - | 21.5 | 43.0 | 64.5 | 86.0 |
| Typhoid fever | | | | |
| Salmonella typhimurium | 80.0 | 160.0 | 240.0 | 320.0 |
| Sarcina lutea | 197.0 | 394.0 | 591.0 | 788.0 |
| Serratia marcescens | 24.2 | 48.4 | 72.6 | 96.8 |
| Shigella dyseteriae - | 22.0 | 44.0 | 66.0 | 88.0 |
| Dysentery | | | | |
| Shigella flexneri - | 17.0 | 34.0 | 51.0 | 68.0 |
| Dysentery | | | | |
| Shigella paradysenteriae | 16.8 | 33.6 | 50.4 | 67.2 |
| Spirillum rubrum | 44.0 | 88.0 | 132.0 | 176.0 |
| Staphylococcus albus | 18.4 | 36.8 | 55.2 | 73.6 |
| Staphylococcus aureus | 26.0 | 57.0 | 78.0 | 104.0 |
| Staphylococcus | 21.6 | 43.2 | 64.8 | 86.4 |
| hemolyticus | | | | |
| Staphylococcus lactis | 61.5 | 123.0 | 184.5 | 246.0 |
| Streptococcus viridans | 20.0 | 40.0 | 60.0 | 80.0 |
| Vibrio comma - Cholera | 33.75 | 67.5 | 101.25 | 135.0 |

Alternatively, energy consumption can be calculated using a targeted UV-C wavelength through the equation below:

E=hc/λ joules

Where:
h=Planck's constant (6.626×10⁻³⁴ J s)
c=Speed of light (2.998×10⁸ m s⁻¹)
λ=Wavelength in m

Once the target energy is identified, the energy consumption (i.e. power) can be calculated using the following equation:

$$P = \frac{Δ E}{Δ t}$$

When the power P (in watts) is calculated, an appropriate power source 60 can be selected.

The cap 50 further includes the switch 72 that causes the microcontroller 62 to generate commands that activate and deactivate the electromagnetic radiation source 68. Alternately, as illustrated in FIG. 6, the switch 72 itself connects and disconnects the power source 60 to the electromagnetic radiation source 68 to control illumination of the electromagnetic radiation source 68. As illustrated in FIG. 3, the switch 72 is disposed on the inner surface of the sidewall 52 of the cap 50. The switch 72 can be an actuated switch such as a micro switch, a spring-loaded switch, or a button switch.

Specifically, the micro switch and/or the spring-loaded switch can be activated based on pressure or an exerted force between the cap 50 and the medication delivery pen 10 during assembly. As illustrated in FIG. 4, upon sensing an increased pressure during assembly, the micro switch 72 sends a signal to the microcontroller 62 to activate the electromagnetic radiation source 68 (capped position). When the cap 50 and the medication delivery pen 10 are disassembled, the pressure is lessened and the micro switch 72 sends a signal to the microcontroller 62 to deactivate the electromagnetic radiation source 68 (uncapped position). The activation and deactivation of the electromagnetic radiation source 68 in this regard can be automatic or instantaneous based on the signaling from the microcontroller 62 or the engagement and disengagement of the micro switch 72.

The switch 72, if provided as a spring-loaded switch, can release a spring force upon receiving an increased pressure during assembly of the cap 50 to the medication delivery pen 10. The spring force provides a one-time activation of the electromagnetic radiation source 68. After a predetermined period of time, the electromagnetic radiation source 68 is deactivated.

The timer 64 can be incorporated into the spring-loaded switch 72, for example, to provide a predetermined time period of photon emission or a time delay before beginning Photon emission. For example, the timer 64 can cause the electromagnetic radiation source 68 to emit photons 70 at a wavelength of 265 nm for up to 120 seconds when the distance between the electromagnetic radiation source 68 and the septum 18 of the medication delivery pen 10 is two inches. The timer 64 can also cooperate with the microcontroller 62 to vary commands for activating and deactivating the electromagnetic radiation source 68.

As illustrated in FIG. 5, the switch 72, when provided as a button switch, can deflect, release a force and/or establish electrical contact with the microcontroller 62 based on an operation, such as a depression, for example, by a user such as the clinician or patient. In this manner, the user is able to control the activation and deactivation of the electromagnetic radiation source 68.

The switch 72 can also be a proximity sensor, a Hall Effect sensor, a photo sensor, an optical sensor and a force sensor. The operation of these sensors are commonly understood by one skilled in the art. The proximity sensor can sense that the cap 50 is disposed on the medication delivery pen 10 and instruct the microcontroller 62 of this condition. Subsequently, the microcontroller 62 can command the electromagnetic radiation source 68 to emit photons 70. When the cap 50 is removed from the medication delivery pen 10, the proximity sensor informs the microcontroller 62 of this condition and the microcontroller commands the electromagnetic radiation source 68 to stop emitting photons 70.

The cap 50 further includes an indicator 66 that displays a plurality of conditions such as indicating when the electromagnetic radiation source 68 is activated or deactivated, when the disinfecting/sterilizing process is complete, and the remaining life of the power source 60. The indicator 66 communicates with the microcontroller 62 to receive a status of one or more of these conditions prior to display. The indicator 66 displays these conditions via a plurality of media commonly known by those skilled in the art such as, for example, colors, symbols and text.

The cap 50 described above provides advantages not realized in the prior art. The cap 50 improves the workflow and convenience of users, such as clinicians or patients, using pen injectors 10. Specifically, the user no longer needs to clean the septum 18 and other surface or portion of the medical device, such as the medication delivery pen 10, with an alcohol swab. This is because the cap 50 alone can disinfect the septum 18 and other surface or portion of the medication delivery pen 10 using photons 70. Accordingly, the user does not need to carry a separate alcohol swab package with the medication delivery pen 10 and does not need to manage extra steps in the process for disinfecting the septum 18 and other surface or portion. Also, the septum 18 and other surface or portion are disinfected more reliably without user error such as ineffective disinfecting or failure to disinfect.

To operate the cap 50 with the medication delivery pen 10, the user simply attaches the cap 50 to the medication delivery pen 10 with or without the universal fitting 40 as described above. Then, either automatically or manually by the user, the electromagnetic radiation source 68 is activated. The electromagnetic radiation source 68 emits photons 70 on the exposed septum 18 of the medication delivery pen 10 to disinfect the septum 18. Other surface or portion of the medication delivery pen 10 is also disinfected. After the disinfection is complete, the cap 50 is subsequently removed. Next, the pen needle is attached to the medicament cartridge 16 of the medication delivery pen 10. The medication delivery pen 10 is now ready for medication delivery.

Upon completion of medication delivery, the pen needle is to be removed from the medicament cartridge 16 and discarded. The septum 18 of the medicament cartridge 16 in the medication delivery pen 10 is now exposed. Next, the user returns and attaches the cap 50 to the medication delivery pen 10. Disinfection of the septum 18 and other surface or portion of the medication delivery pen 10 resumes similarly as described above. This disinfection process can be repeated between multiple injections of the medication delivery pen 10.

In a simpler implementation as mentioned above and illustrated in FIG. 6, the button switch 72 and a current limiting resistor 74 can control the electric power from the power source 60 to the electromagnetic radiation source 68 directly, without the microcontroller 62. In this case, the user controls the duration of the disinfection by the length of time that the button switch 72 is operated, activated or depressed, for example. That is, when the switch 72 is in operation, or depressed, the electromagnetic radiation source 68 uses the electric power from the power source 60 to illuminate the electromagnetic radiation source 68. When the switch 72 is not in operation, or not depressed, the electromagnetic radiation source 68 does not use the electric power from the power source 60. As a result, no disinfection takes place.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. Any of the embodiments and/or elements disclosed herein may be combined with one another to form various additional embodiments not specifically disclosed, as long as they do not contradict each other. Accordingly, additional embodiments are possible and are intended to be encompassed within this specification and the scope of the invention. The specification describes specific examples to accomplish a more general goal that may be accomplished in another way.

As used in this application, the terms “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately” are understood by those of ordinary skill to refer to reasonable ranges outside of the given value, for example, general tolerances associated with manufacturing, assembly, and use of the described embodiments.

What is claimed is:

1. A cap configured to disinfect a medical device or portion thereof, the cap comprising:
a power source that provides electric power;
an electromagnetic radiation source that uses the electric power received from the power source to emit photons for disinfection; and
a switch that is configured to be operated by user action; wherein
upon activation of the switch, the electric power from the power source is applied to the electromagnetic radiation source to radiate photons onto the medical device or portion thereof.

2. The cap of claim 1, wherein the power source comprises a battery.

3. The cap of claim 2, wherein the battery is disposed opposite to a portion of the medical device when the cap is attached to the medical device.

4. The cap of claim 1, wherein the power source is disposed along a sidewall of the cap.

5. The cap of claim 1, wherein the electromagnetic radiation comprises one or more light emitting diodes (LEDs).

6. The cap of claim 1, wherein
the electromagnetic radiation source emits light in a bandwidth to disinfect a portion of the medical device; and
the portion of the medical device is a surface of the medical device.

7. The cap of claim 1, wherein the switch comprises a spring-loaded switch or a button switch or a sensor.

8. A medication pen needle assembly, comprising:
the cap of claim 1;
the medical device comprising a medication delivery pen; and
a universal fitting disposed between the cap and the medication delivery pen to secure the cap onto the medication delivery pen.

9. The medication pen needle assembly of claim 8, wherein the universal fitting includes a ring, ribs, pleats, scales, a spring-loaded button, prongs, or a telescoping pole.

10. A medication pen needle assembly, comprising:
the cap of claim 1;
the medical device comprising a medication delivery pen; and
a pen needle attached to the medication delivery pen, wherein
the electric power from the power source is applied to the electromagnetic radiation source to radiate photons on a needle of the pen needle.

11. The cap of claim 1, wherein the cap is replaceable.

12. The cap of claim 1, wherein the power source is replaceable.

13. The cap of claim 1, wherein the power source is rechargeable via solar energy, motion or wired electric power.

14. A cap configured to disinfect a medical device or portion thereof, the cap comprising:
a power source that provides power to a microcontroller, the microcontroller sensing and controlling the operation of the cap;
an electromagnetic radiation source that radiates photons on the medical device or portion thereof for disinfection under control of the microcontroller; and
a switch that causes the microcontroller to activate and deactivate the electromagnetic radiation source.

15. The cap of claim 14, wherein the switch comprises a micro switch, a proximity sensor, a Hall effect sensor, a photosensor, an optical sensor or a force sensor.

16. The cap of claim 14, further comprising an indicator that indicates at least one of whether the electromagnetic radiation source is activated, whether a disinfection process is complete, and a remaining life of the power source.

17. The cap of claim 14, further comprising a timer that controls at least one of a time delay and a duration of the radiation.

18. A method for disinfecting a medical device or portion thereof with a cap, the method comprising:
disposing an electromagnetic radiation source on an inner surface of the cap;
securing the cap to the medical device;
activating the electromagnetic radiation source to emit photons to disinfect the medical device or portion thereof; and
exposing the medical device or portion thereof to photons from the electromagnetic radiation source.

19. The method of claim 18, further comprising removing a pen needle from the medical device comprising a medication delivery pen to expose a septum of the medical device prior to securing the cap.

20. The method of claim 18, wherein when the cap is secured onto the medical device comprising a medication delivery pen, the electromagnetic radiation source is automatically activated.

21. The method of claim 18, further comprising activating a switch to activate the electromagnetic radiation source.

22. The method of claim 18, wherein the electromagnetic radiation emits ultraviolet light in a bandwidth of 250 nm to 280 nm to disinfect the medical device or portion thereof.

23. The method of claim 18, further comprising displaying, via an indicator, at least one of whether the electromagnetic radiation source is activated, whether a disinfection process is complete, and a remaining life of the power source.

24. The method of claim 18, further comprising controlling, via a timer, at least one of a time delay and a duration of photons emission.

25. The method of claim 18, further comprising disposing a universal fitting between the cap and the medical device comprising a medication delivery pen to secure the cap.

26. The method of claim 18, further comprising recharging a power source of the cap.
